(19) United States
(12) Patent Application Publication
(10) Pub. No.: US 2005/0233970 A1
(43) Pub. Date: Oct. 20, 2005

(54) Methods for treating long QT syndrome

(75) Inventor: Marc B. Garnick, Brookline, MA (US)

Correspondence Address: LAHIVE & COCKFIELD, LLP., 28 STATE STREET, BOSTON, MA, 02109, US

(73) Assignee: Praecis Pharmaceuticals, Inc., Waltham, MA (US)

(21) Appl. No.: 11/088,462

(22) Filed: Mar. 23, 2005

Related U.S. Application Data
(60) Provisional application No. 60/555,946, filed on Mar. 23, 2004.

(51) Int. Cl. A61K 38/09; A61K 31/55; A61K 31/56

(52) U.S. Cl. 514/15; 514/16; 514/171

(57) ABSTRACT

The present invention provides compositions and methods for treating QT prolongation in a subject in need thereof. The method comprises the step of administering to the subject a therapeutically effective amount of an agent which increases the androgen level of the subject.

### RELATED APPLICATIONS

This application claims priority to U.S. Provisional Patent Application Ser. No. 60/555,946, filed on Mar. 23, 2004, the entire contents of which are incorporated herein by reference.

### BACKGROUND OF THE INVENTION

The measurement of the contraction of the heart on an electrocardiogram (ECG) produces a waveform with characteristic elements which correspond to the various stages of contraction. One feature of an ECG is referred to as the QT interval, which represents the time period between the initiation of ventricular depolarization and completion of repolarization. The QT interval varies with the heart rate, age and gender. For example, the QT interval decreases with increasing heart rate. Men generally have shorter QT intervals than women.

Under certain circumstances, the QT interval can be prolonged, increasing the risk of a potentially fatal cardiac arrhythmia referred to as “Torsade des pointes” (TdP). TdP results in the inability of the heart to contract effectively, leading to a decrease in bloodflow to periphery, including the brain, and syncope or sudden death. In rare cases, a prolonged QT interval is congenital and usually inherited. In other cases, prolongation of the QT interval is the result of a neurological disorder, such as stroke. Most frequently, prolonged QT interval is caused by certain medications.

Treatment for prolonged QT interval, also referred to as “long QT syndrome”, is currently limited to beta blockers, surgery and implantation of a defibrillator. There is, therefore, a need for a new therapeutic agents and methods for treating and/or preventing QT prolongation.

### SUMMARY OF THE INVENTION

The present invention provides compositions and methods for treating QT prolongation in a subject in need thereof. The method comprises the step of administering to the subject a therapeutically effective amount of an agent which increases the androgen level of the subject. The agent can be one or more androgens themselves, such as, for example, testosterone or dihydrotestosterone. The subject can be any patient who has a prolonged QT interval, i.e., suffers from long QT syndrome, who is at risk for developing QT prolongation, or is suffering from a condition associated with, or caused by, QT prolongation. Suitable subjects include those who have congenital long QT syndrome, acquired long QT syndrome or who are taking or are scheduled to begin taking a medication known to cause QT prolongation.

### DESCRIPTION OF THE INVENTION

The present invention relates to the observation, described below, that certain drugs used in the hormonal therapy of prostate cancer result in an increase in the QT interval. These drugs cause a reduction in testosterone to castrate levels or inhibit the interaction of androgens with the androgen receptor and point to a cardioprotective effect of androgens. The invention thus provides a method of treating a subject having a prolonged QT interval or at risk for developing QT prolongation by increasing the plasma androgen level of the subject. The method comprises the step of administering to the subject a therapeutically effective amount of an agent which increases the plasma androgen level of the subject. The subject can be a mammal, preferably a primate and, most preferably, is a human. The subject can be male or female.

In one embodiment, the subject suffers from congenital long QT syndrome or an acquired long QT syndrome. For these subjects, the method of the invention can be used to reduce the QT interval, or the frequency of episodes of QT interval prolongation.

In another embodiment, the subject is at risk for developing QT prolongation. The term “subject at risk for developing QT prolongation”, refers to a subject taking a drug known to cause QT prolongation, a subject who is hypogonadal, i.e., a subject having an abnormally low serum testosterone level, or a subject having a history which suggests the possibility of episodes of prolonged QT interval. Subjects have such a history if they have, for example, previously experienced one or more of the following: arrhythmia, ventricular arrhythmia, cardiac arrhythmia, abnormal ECG, palpitation, ventricular tachycardia, cardiac arrest, syncope, hypotension, postural hypotension, seizure, grand mal seizure, transient ischaemic attack, Torsade de Pointes, cardiac fibrillation, convulsions, ventricular fibrillation, ventricular flutter and ventricular trigeminy.

For example, the subject can be, or is expected to be, treated with a medication known to cause QT prolongation. Such medications include, but are not limited to, albuterol, alfuzosin, amantadine, amiodarone, arsenic trioxide, atomoxetine, azithromycin, bepridil, chloral hydrate, chloroquine, chlorpromazine, cisapride, clarithromycin, cocaine, disopyramide, dobutamine, dofetilide, dolasetron, domperidone, dopamine, droperidol, ephedrine, epinephrine, erythromycin, felbamate, fenfluramine, flecainide, foscamet, fosphenytoin, gatifloxacin, granisetron, halofantrine, haloperidol, ibutilide, indapamide, isoproterenol, isradipine, levalbuterol, levofloxacin, levomethadyl, lithium, mesoridazine, metaproterenol, methadone, methylphenidate, midodrine, moexipril, moexipril/hydrochlorothiazide, moxifloxacin, nicardipine, norepinephrine, octreotide, ondansetron, pentamidine, phentermine, phenylephrine, phenylpropanolamine, pimozide, procainamide, pseudoephedrine, quetiapine, quinidine, risperidone, ritodrine, salmeterol, sibutramine, sotalol, sparfloxacin, tacrolimus, tamoxifen, telithromycin, terbutaline, thioridazine, tizanidine, vardenafil, venlafaxine, voriconazole, and ziprasidone. In this embodiment, the subject can have a congenital or acquired long QT syndrome, or can be free of long QT syndrome, but potentially susceptible to drug-induced QT prolongation, such as a subject at risk for developing QT prolongation or a subject suffering from hypogonadism.

The subject can also be suffering from a condition associated with QT prolongation, such as arrhythmia, ventricular arrhythmia, cardiac arrhythmia, abnormal ECG, palpitation, ventricular tachycardia, cardiac arrest, syncope, hypotension, postural hypotension, seizure, grand mal seizure, transient ischaemic attack, Torsade de Pointes, cardiac fibrillation, convulsions, ventricular fibrillation, ventricular flutter or ventricular trigeminy. The condition can also be arrhythmia due to QT alterations. In embodiments in which the subject suffers from such a condition, the method preferably comprises the acute administration of the agent which increases the serum androgen level of the subject, i.e., the agent is administered via a route which is suitable for rapidly increasing the serum androgen level of the subject, for example, within minutes or hours. When the condition is life-threatening, for example, it is preferred to increase the serum androgen level as rapidly as is practicable. For example, parenteral administration of the agent, such as intravenous, intramuscular, subcutaneous or intraperitoneal injection of the agent can be used to effect a rapid increase in the serum androgen level.

The agent which increases the subject's serum androgen level can be any agent capable of increasing serum androgen levels in the subject. The agent can be, for example, LHRH or an LHRH agonist, such as leuprolide, goserelin, histrelin, triptorelin or others known in the art. Such compounds cause a transient increase in testosterone levels in men, before ultimately causing chemical castration. When the agent is an LHRH agonist, the agent is preferably administered in such a way as to enhance testosterone production in the subject and then discontinued prior to onset of testosterone reduction. The agent can also be luteinizing hormone (LH), human chorionic gonadotropin (hCG) or any agent which stimulates LH release. Agents such as LHRH, LHRH agonists, LH and hCG are preferably used when the subject is male.

The subject's serum testosterone level can be increased to at least about 100 ng/dL. Preferably, the subject's serum testosterone level is increased to about 300 ng/dL or greater. In one embodiment, the subject is a woman, and the subject's serum testosterone level is increased to about 100 ng/dL or greater. In one embodiment, the subject' serum testosterone level is increased to a value in the range from about 200 ng/dL to about 1100 ng/dL or greater, preferably from about 300 ng/dL to about 1000 ng/dL. In one embodiment, the serum testosterone is increased to a value from about 1000 ng/dL to about 2500 ng/dL or from about 2500 ng/dL to about 5000 ng/dL. In another embodiment, the subjects serum testosterone level is increased by at least about 50 ng/dL, 100 ng/dL, 200 ng/dL, 300 ng/dL or 500 ng/dL. In certain embodiments, the uincrease in serum testosterone levels can be even greater, for example, at least about 600 ng/dL, 700 ng/dL, 800 ng/dL, 900 ng/dL, 1000 ng/dL or 1500 ng/dL. The achievement of such serum testosterone levels can be monitored using testosterone assays which are known in the art. A suitable dose and dosing schedule of the agent for reaching the desired serum testosterone level can be determined by one of skill in the art based upon the agent to be administered, the route of administration, the subject's baseline serum testosterone level (prior to treatment) and the subject's age and weight. The subject's serum androgen level can be monitored for achievement of the desired level using methods known in the art.

The agent is, preferably, an androgen, such as testosterone or dihydrotestosterone, or a testosterone derivative that is converted to testosterone or dihydrotestosterone in vivo, such as, for example, another steroid such as 4-androstenediol or androstenedione. In a particularly preferred embodiment, the agent is testosterone or a testosterone derivative with androgenic properties, including testosterone cypionate, testosterone enanthate, testosterone undecanoate, and 17-alpha-alkyltestosterone, including 17-alpha-methyltestosterone and 17-alpha-fluoxytestosterone.

The agent which increases serum testosterone levels is incorporated into a pharmaceutical composition suitable for administration to a subject. Such a pharmaceutical composition comprises the agent an a pharmaceutically acceptable carrier. In a preferred embodiment, the pharmaceutical composition comprises testosterone and a pharmaceutically acceptable carrier.

The agent which increases serum testosterone levels can be administered via any route consistent with amount of agent to be administered and the desired time scale for administration. For example, the agent can be administered via intravenous, subcutaneous, intraperitoneal, or intramuscular injection; or transdermal, topical, rectal, vaginal or buccal administration.

As used herein “pharmaceutically acceptable carrier” includes any and all solvents, dispersion media, coatings, antibacterial and antifungal agents, isotonic and absorption delaying agents, and the like that are physiologically compatible. In one embodiment, the carrier is suitable for parenteral administration or for administration via inhalation. Preferably, the carrier is suitable for administration into the central nervous system (e.g., intraspinally or intracerebrally). Alternatively, the carrier can be suitable for intravenous, subcutaneous, intraperitoneal, intramuscular, transdermal, topical, rectal, vaginal or buccal administration. In another embodiment, the carrier is suitable for oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as manitol, sorbitol, or sodium chloride in the composition. Prolonged absorption of the injectable compositions can be brought about by including in the composition an agent which delays absorption, for example, monostearate salts and gelatin. Moreover, the compounds of the invention can be administered in a time release formulation, for example in a composition which includes a slow release polymer. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are patented or generally known to those skilled in the art.

Sterile injectable solutions can be prepared by incorporating the agent in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Suitable pharmaceutical compositions for use in the method of the invention include injectable solutions of an LHRH agonist, including short-acting compositions and depot compositions. Suitable depot compositions include those designed to release the LHRH agonist over a period of about one month or less, such as the one-month depot formulations of Lupron™ (TAP Pharmaceutical Products, Inc.) and Zoladex™ (Astra Zeneca).

Suitable pharmaceutical compositions for use in the method of the invention further include compositions comprising one or more androgens, such as testosterone or dihydrotestosterone, in a suitable pharmaceutical carrier. A variety of such compositions are known in the art, including Androgel™ (Solvay Pharmaceuticals), Testim™ (Auxilium Pharmaceuticals), Striant™ (Columbia Laboratories), Testoderm™ (Alza Corporation), and Androderm™ (GlaxoSmthKline). Other suitable pharmaceutical compositions for use in the present method include those described in the following published PCT applications: WO 03/026649; WO 02/066018; WO 02/055020; WO 03/61664; WO 98/34621; WO 01/87316; WO 99/65228; WO 00/71133; WO 03/49732; WO 02/17967; and WO 00/19975. The contents of each of the foregoing references are incorporated by reference herein in their entirety.

### EXAMPLE

Three prospective, randomized clinical studies were conducted which compared the efficacy of abarelix, a GnRH antagonist, to the existing and widely used hormonal therapies: 1) goserelin plus bicalutamide, 2) leuprolide plus bicalutamide, and 3) leuprolide alone. These effective strategies to induce androgen depletion allowed the opportunity to evaluate the effect of induced androgen deficiency on the QT interval by obtaining an ECG prior to and during treatment with each of the hormonal therapies.^{(F)}

Methods

Study I, conducted in Europe, was a randomized (1:1), active controlled evaluation of abarelix versus goserelin plus bicalutamide in 177 patients with Stage D1 or D2 prostate cancer, or rising PSA titer following definitive local therapy for prostate cancer. Patients were at least 18 years old; had histologically confirmed prostate cancer; were previously untreated with hormonal therapy; and had an ECOG performance status of ≦2, life expectancy of ≧3 months and serum testosterone of ≧220 ng/mL and ≦2×ULN. Goserelin was given as a 3.6 mg subcutaneous dose every 28 days for 48 weeks; bicalutamide was given in a dose of 50 mg daily, starting with the administration of goserelin. Abarelix was given as a dose of 100 mg IM on days 1, 15, 29 and every 28 days thereafter for 48 weeks. Prospectively, a standard 12-lead ECG was performed at baseline and on days 85 and 337. Plasma testosterone samples were obtained at the same time. All ECG's were analyzed in a blinded fashion to treatment allocation. Each ECG was analyzed in comparison to its respective baseline for heart rate, QRS duration, PR interval, QT interval, and QTc. Concomitant medicines and prior medical history was recorded on all patients. Patients with a baseline QT of >450 msec were to be excluded from the study. If a patient experienced a QTcB of ≧500 msec while on study, therapy was to be discontinued.

Studies II and III, conducted in North America, were randomized (2:1, abarelix:active control) trials that compared abarelix to either leuprolide monotherapy (Study II) or leuprolide plus bicalutamide (Study III) in patients having one or more of the following: Stage D1 or Stage D2 prostate cancer, rising PSA following definitive localized therapy, or were candidates for neoadjuvant or intermittent hormonal therapy. Abarelix was given in an identical fashion to Study I; leuprolide was given at a dose of 7.5 mg IM every 28 days; bicalutamide was given in a dose of 50 mg daily starting with the administration of leuprolide. Studies II and III required an ECG at baseline and on day 169 and, if the patient continued, on day 365. Plasma testosterone and dihydrotestosterone samples were obtained at the same time. Unlike Study I, Studies II and III did not have any prospectively defined criteria for QTc exclusion or withdrawal based upon the QT interval. Based upon the QT observations obtained from Study I, an analysis of ECGs obtained as part of Study II and Study III was undertaken. A total of 299 patients had a complete set of ECG's and form the basis for this ECG analysis. Data for the abarelix arms were pooled from Studies II and III. The ECG machine-calculated heart rate and QT interval were entered into a database. QTcB and QTcF were calculated utilizing standard formulas.^{(F)}

Plasma Testosterone and Dihydrotestosterone Analysis Measurements

Laboratory analysis of testosterone and dihydrotestosterone were determined using certified central laboratories and standard radioimmunoessay methods.

QTc Analysis

QTc evaluations included measurement of baseline, post baseline, on-therapy and change from baseline in QTcB and QTcF in each treatment group. They are the focus of this report. Study I also included an analysis of concomitant medicines during the conduct of the study and an assessment of any potential influence on the QT interval. A blinded assessment of cardiovascular adverse events on therapy was performed.

Statistical Methods

ECG measurement of QTcB and QTcF were calculated based on standard QT and heart rate measurements. T-tests were used to evaluate intra as well as inter-treatment change from baseline for each treatment group. Comparisons between treatment groups were performed in a pair wise manner.

Results

Patient Characteristics

A total of 465 elderly men with advanced prostate cancer participated in this study. The number of patients with baseline and on-treatment ECG evaluations are presented in Tables 1A (Study I), and 1B (Studies II and III), along with demographic data. The demographic profile and age are typical of men with advanced prostate cancer.

Serum Androgen Levels

Table 2 sets forth the effects of treatment on testosterone levels. In all cases, the median testosterone level was below 50 ng/dl (considered castration levels). In studies II and III, dihydrotestosterone levels were also determined. After 169 days of therapy, dihydrotestosterone levels had decreased to between 25-30 pg/ml for the three treatment groups, consistent with castration.

Electrocardiographic Measurement of QTc

In Study I, a total of 177 patients were randomized; of these, 166 [goserelin plus bicalutamide—(N=86) or abarelix—(N=82)] had baseline and on-treatment QTC data. The baseline values and changes in QT interval during therapy are presented in Tables 3A and 3B. As shown in Table 3A, the combination of goserelin plus bicalutamide increased baseline QTcB by an average of 16.7 msec while treatment with abarelix increased QTcB by an average of 13.3 msec (both p<0.001 vs. baseline, respectively). The QTc change between groups was not significant. The Fredericia correction for QTc is also presented in Table 3A, and yielded highly significant results for each treatment group compared to baseline (p<0.001). The between-group comparison was modestly significant (p=0.018) for a greater increase in QTcF in goserelin plus bicalutamide-assigned patients.

The QTc measurements of the randomized treatment groups in Studies II and III are presented in Table 3B. Based on the Bazett correction, the QTcB increased during therapy by 20.0 msec, 9.4 msec and 13.8 msec in patients randomized to leuprolide, leuprolide plus bicalutamide and abarelix, respectively. All the increases compared to their respective baselines were significant (p<0.001, p=0.018, and p<0.001, respectively). Comparison between groups revealed that leuprolide therapy alone had a marginally greater change in QTcB at baseline than leuprolide plus bicalutamide or abarelix, respectively (p=0.049). The same analyses using the Fredericia correction are consistent in that all groups increased QTcF during therapy by 17.6, 9.9 and 12.8 msec, respectively. All three QTcF changes compared to baseline were highly statistically significant (all p≦0.006; Table 3B). QTcB or QTcF values ≧500 msec occurred rarely and were equally distributed among treatment groups, and were without clinical consequence.

In summary, in three separate trials of four therapeutic strategies of hormonal therapy of advanced prostate cancer, testosterone levels were reduced to castrate levels. During the same interval, compared to baseline, QTc increases occurred consistently in all treatment groups. Since ECGs were collected at trough, immediately prior to the next depot injection, the QTc increases observed are less likely a direct effect, but rather due to the induction of medical castration. There were no effects on heart rate, PR or QRS intervals.

Standard adverse event reporting was utilized throughout all clinical trials. There were no cases of torsades de pointes ventricular tachycardia seen. The incidences of cardiovascular events that even remotely suggest proarrhythmia were recorded and were of low frequency (palpitations, tachycardia, syncope, seizure, cardiac arrest). The total rate of such events was small (<5%) and similar between treatment groups. There were no reported sudden deaths. A review of concomitant medicines in Study I was undertaken to assess any potential confounding association with QT prolongation and to assess whether patients who exhibited the greatest QT prolongation might have received drugs known to prolong QT interval. No relationship was observed and such QT prolonging medications were used in <10% of patients.

Discussion

The goal of current therapies for advanced prostate carcinoma is the induction of androgen deficiency. Original ECG data are presented here on four clinical trials using alternative treatment therapies that accomplish that goal. In each case, the reduction of serum testosterone by 89-97% to castrate levels over 5-11 months resulted in ECG evidence of QT increases of 9-20 msec or 10-18 msec, whether Bazett or Fredericia formula corrections were used. Plasma testosterone and dihydrotestosterone were measured at the same time ECG's were obtained, confirming that androgen deficiency was associated with the QTc increases. This study provides new and original data in patients that androgen deficiency, or other factors resulting from the state of androgen deficiency, are associated with QT prolongation. This result implies that testosterone or other androgens play a significant role in cardiac repolarization. The magnitude of the QT prolongation documented in these studies is comparable to the QT increase observed with low dose sotalol or dofetilide, two commonly used Ikr blockers for treating arrhythmias (Reiffel J A. Am Heart J 1998;135:551-556; Torp-Pederson C et al. Eur Heart J 2000; 21: 1204-1206). The magnitude of the QT prolongation reported here is also consistent with QT prolongation reported in preliminary case observations in orchiectomized males or decreases in QT seen in females with virilization syndromes (Bidoggia H. et al. Am Heart J 2000;140:678-683).

QT prolongation is associated with a specific life-threatening ventricular arrhythmia, torsades de pointes ventricular tachycardia. Although a number of ion channels are responsible for ventricular repolarization, the ion channel most associated with adverse drug interactions leading to proarrhythmia is the rapid component of the delayed rectifier potassium channel, the Ikr channel. Small changes in chemical structure can result in dramatic changes in effect on the Ikr channel and, therefore, on the QT interval. An example of the stereospecificity of closely associated drugs and their relative effect on Ikr is terfenadine, an antihistamine that prolongs the QT interval (Pratt CM et al.; Am Heart J 1996;131:472-480). Its acid metabolite, fexofenadine, although chemically very similar, has no effect on Ikr and thus on the QTc (Pratt C M et al. Am J Cardiol 1999; 83:1451-1454). In the clinical study presented here, four approved therapies of advanced prostate cancer all significantly prolong the QT interval. Goserelin, bicalutamide, abarelix and leuprolide have varied mechanisms of action, but all produce actual or functional testosterone deficiency. These four treatments not only have varied mechanisms (LHRH agonist, antiandrogens, GnRH antagonist) but also dissimilar chemical structures. Furthermore, the effects on the QT interval were measured at the time of trough drug levels, just prior to the next depot injection. Therefore, although it is possible these therapies might have a direct effect on Ikr channel expression, it seems more likely that they exert their effect through testosterone itself or some other factor which modulates it. Regardless of the specific mechanism, these data support an important role, direct or indirect, of androgens such as dihydrotestosterone and testosterone on Ikr channel expression, and by implication, the risk of torsades. In adults, females have a QT interval approximately 10 msec longer than males. This difference is not present in newborns, but presumably appears by puberty (Pham TV, et al., Circulation 2001;103:2207-2212).

This study provides a new perspective on the observation that females are more susceptible to drugs that prolong repolarization compared to males. This differential risk for proarrhythmia exists regardless of whether the offending agent is a cardiovascular drug such as sotalol or a non-cardiac drug such as cisapride (Bednar M M, et al., Am J Cardiol 2002; 89:1316-1319). In fact, a two to four-fold increase of risk in females (compared to males) to develop torsades with drugs that prolong repolarization is a consistent finding for all drug classes that increase the QT interval. When all risk factors for torsades are analyzed by regression analysis, female gender is consistently one of the strongest predictors of increased torsades risk (Lehmann M H, et al., Circulation 1996; 94: 2535-2541; Pratt C M, et al., Am Heart J 1996;131:472-480). The data presented here are consistent with a protective role of testosterone, and its active metabolic dihydrotestosterone.

This study suggests a previously unappreciated protective role of androgens, directly or indirectly, on the QT interval and inferentially on the risk of developing torsades.

TABLE 1A
Demographics: Study I

| | Goserelin plus Bicalutamide | Abarelix |
|---|---|---|
| | N = 84 | N = 82 |
| Mean Age (yr) | 69 | 70 |
| MI* | 6 | 7 |
| CHF | 0 | 2 |
| Hypertension | 39 | 44 |
| Diabetes | 8 | 10 |
| Smoking | 1 | 0 |
| Cholesterol Elevation | 17 | 16 |
| LBBB/BBB^{§} | 1 | 0 |
| Angina | 8 | 4 |
| CAD† | 5 | 6 |
| Atrial fibrillation | 0 | 1 |
| Arrhythmia | 1 | 6 |
| Pacemaker | 1 | 2 |
| Family history | 0 | 0 |
| Cardiac medication | 19 | 23 |

*All data is expressed as percent of patients, except age.
†Coronary artery disease with or with out intervention.
^{§}BBB = bundle-branch block; LBBB = left bundle-branch block

TABLE 1B
Demographics: Studies II and III

| | Treatment Group | | |
|---|---|---|---|
| | Leuprolide | Leuprolide plus Bicalutamide | Abarelix |
| | N = 51 | N = 39 | N = 209 |
| Mean Age | 74 | 72 | 72 |
| MI* | 12 | 5 | 10 |
| CHF | 35 | 41 | 49 |
| Hypertension | 35 | 41 | 49 |
| Diabetes | 12 | 13 | 14 |
| Smoking | 8 | 5 | 8 |
| Cholesterol | 16 | 31 | 29 |
| Elevation | | | |
| LBBB/BBB | 0 | 3 | 1 |
| Angina | 2 | 10 | 5 |
| CAD† | 20 | 21 | 19 |
| Atrial fibrillation | 4 | 0 | 8 |
| Arrhythmia | 0 | 10 | 3 |
| Pacemaker | 2 | 3 | 1 |
| Family history | 0 | 0 | 0 |
| Cardiac meds | 6 | 28 | 16 |

*All data is expressed as percent of patients, except age.
†Coronary artery disease with or with out intervention.

TABLE 2
Testosterone Levels (ng/dL) on Study Days When ECG was Evaluated
Study I

| | Goserelin plus | | | |
|---|---|---|---|---|
| Study | Bicalutamide | | Abarelix | |
| Day | Median | IQR* | Median | IQR* |
| Baseline | 347 | (265, 447) | 316 | (254, 444) |
| Day 85 | 23 | (17, 32) | 23 | (20, 35) |
| Day 337 | 26 | (17, 35) | 35 | (23, 49) |

Studies II and III

| | | | Leuprolide + | | | |
|---|---|---|---|---|---|---|
| Study | Leuprolide | | Bicalutamide | | Abarelix | |
| Day | Median | IQR* | Median | IQR* | Median | IQR* |
| Base- | 342 | (280, 446) | 358 | (275, 427) | 361 | (289, 453) |
| line | | | | | | |
| Day | 9 | (8, 20) | 13 | (8, 19) | 14 | (8, 24) |
| 169 | | | | | | |

*IQR = interquartile interval

TABLE 3A
Study I: QTc Changes Associated with Two Alternative Therapies for
Advanced Prostate Cancer.

| | | Randomized | | |
|---|---|---|---|---|
| | | Treatment Group | | |
| | | (mean msec ± S.D.) | | |
| | | Goserelin | | |
| | | plus | | |
| | | Bicalutamide | Abarelix | P value# |
| QTcB | Baseline | 411.4 (26.5) | 413.5 (26.8) | |
| | On Therapy | 428.0 (27.9) | 426.8 (22.9) | |
| | Change from Baseline | 16.7 (20.6) | 13.3 (18.4) | 0.26 |
| | P value* | <0.001 | <0.001 | |
| QTcF | Baseline | 404.0 (22.7) | 404.6 (23.5) | |
| | On Therapy | 422.4 (26.4) | 419.5 (21.3) | |
| | Change from Baseline | 18.3 (18.1) | 12.0 (16.2) | 0.018 |
| | P value* | <0.001 | <0.001 | |

Abbreviations: QTcB = Bazett correction; QTcF = Fredericia correction; SD = standard deviation.
*t-test, intra-treatment change from baseline

TABLE 3B
Studies II and III: QTc Changes Associated with Three Alternative
Therapies for Prostate Cancer

| | | Randomized Treatment Group | | |
|---|---|---|---|---|
| | | (median ± SD) | | |
| | | | Leuprolide plus | |
| | | Leuprolide | Bicalutamide | Abarelix |
| | | (N = 51) | (N = 39) | (N = 209) |
| QTcB | Baseline | 421.6 (29.4) | 418.7 (29.3) | 417.9 (28.6) |
| | On Therapy* | 441.6 (25.8) | 428.1 (20.9) | 431.7 (29.8) |
| | Change from | 20.0 (26.0) | 9.4 (23.7) | 13.8 (24.6) |
| | Baseline | | | |
| | P value* | 0.001 | 0.018 | <0.001 |
| QTcF | Baseline | 414.5 (26.0) | 414.3 (26.9) | 410.6 (28.1) |
| | On Therapy | 432.1 (23.6) | 424.2 (19.8) | 423.4 (28.0) |
| | Change from | 17.6 (25.0) | 9.9 (21.3) | 12.8 (23.1) |
| | Baseline | | | |
| | P value* | <0.00 1 | 0.006 | <0.00 1 |

Abbreviations as before.
*t-test, intra-treatment change from baseline
Intergroup comparisons for QTcB, QTcF, respectively; abarelix vs. leuprolide, p = 0.11, 0.19; abarelix vs. leuprolide plus bicalutamide, p = 0.30, 0.47; leuprolide vs. leuprolide plus bicalutamide, p = 0.49, 0.13.

1. A method for treating QT prolongation in a subject in need thereof comprising the step of administering to the subject a therapeutically effective amount of an agent which increases the serum androgen level of the subject.

2. The method of claim 1 wherein the agent comprises one or more androgens, LHRH or an LHRH agonist.

3. The method of claim 1 wherein the subject has congenital long QT syndrome, acquired long QT syndrome or is at risk for developing QT prolongation.

4. The method of claim 3 wherein the subject is hypogonadal or the subject is taking a medication known to cause QT prolongation.

5. The method of claim 1 wherein the agent is testosterone, dihydrotestosterone or a testosterone derivative.

6. The method of claim 5 wherein the agent is testosterone.

7. The method of claim 1 wherein the subject's serum testosterone level is increased to at least about 100 ng/dL.

8. The method of claim 1 wherein the subject is suffering from a condition associated with QT prolongation.

9. The method of claim 8 wherein the condition associated with QT prolongation is selected from the group consisting of arrhythmia, ventricular arrhythmia, cardiac arrhythmia, abnormal ECG, palpitation, ventricular tachycardia, cardiac arrest, syncope, hypotension, postural hypotension, seizure, grand mal seizure, transient ischaemic attack, Torsade de Pointes, cardiac fibrillation, convulsions, ventricular fibrillation, ventricular flutter and ventricular trigeminy.

10. The method of claim 9 wherein the agent which increases the serum androgen level of the subject is administered parenterally.

11. The method of claim 10 wherein the agent is administered by intravenous injection, subcutaneous injection, intramuscular injection or intraperitoneal injection.

12. The method of claim 11 wherein the agent is testosterone, dihydroxytestosterone or a combination thereof.

13. The method of claim 13 wherein the agent is administered in a pharmaceutical composition.
